(19) United States
(12) Patent Application Publication
(10) Pub. No.: US 2026/0083890 A1
(43) Pub. Date: Mar. 26, 2026

(54) PERFUSION PUMP WITH AUTOMATIC OCCLUSION DETECTION AND CONTROLLED ADJUSTMENT

(71) Applicant: Maquet Cardiopulmonary GmbH, Rastatt (DE)

(72) Inventors: Jonas HEIBERGER, Oberkirch (DE); Jens SCHRÖTER, Karlsbad (DE); Frank STICKEL, Villingen-Schwenningen (DE)

(21) Appl. No.: 19/409,336

(22) Filed: Dec. 4, 2025

Related U.S. Application Data
(63) Continuation of application No. PCT/EP2024/065251, filed on Jun. 4, 2024.
(60) Provisional application No. 63/506,173, filed on Jun. 5, 2023.

(51) Int. Cl. A61M 1/36

(52) U.S. Cl. CPC A61M 1/3601; A61M 1/367; A61M 2205/103; A61M 2205/3327; A61M 2205/3331; A61M 2205/50

(57) ABSTRACT

A roller pump including a pump head operably connected to be driven by a pump drive, wherein the pump head includes: a pump stator; a pump rotor disposed to rotate within the pump stator, wherein the pump rotor includes one or more rollers, wherein each roller is connected to a respective roller block that is moveable radially with respect to a central axis of the pump head; an occlusion adjustment mechanism, connected to the roller block of each roller, wherein the occlusion adjustment mechanism operates to move each roller block radially with respect to the central axis of the pump head in order to compress and partially occlude a compressible fluid conduit disposed within the raceway; and a sensor arrangement configured to measure a pressure exerted by the roller block of at least one roller when the compressible fluid conduit is compressed by one or more of the rollers.

### CROSS-REFERENCE TO RELATED APPLICATIONS

This application is a continuation under 35 U.S.C. § 120 of International Application No. PCT/EP2024/065251, filed Jun. 4, 2024, which claims the benefit of U.S. Provisional Application No. 63/506,173, filed Jun. 5, 2023. Each of the above-referenced patent applications is incorporated by reference in its entirety.

### FIELD OF THE DISCLOSURE

The present disclosure pertains broadly to the field of roller pumps used in medical devices such as heart-lung machines, ECMO machines, other cardiopulmonary bypass machines, and extracorporeal circulation machines employed for dialysis. More specifically, the field of this disclosure may be construed as directed to an occlusion mechanism, for use in medical devices such as roller pumps for heart-lung machines, ECMO machines, other cardiopulmonary bypass machines, and extracorporeal circulation machines employed for dialysis.

### DESCRIPTION OF RELATED ART

Roller pumps, also known as peristaltic pumps, are used in medicine and surgery to circulate blood in extracorporeal circulation machines, such as dialysis machines, and in cardiopulmonary bypass machines, such as heart-lung machines and extracorporeal membrane oxygenation (ECMO) machines. Roller pumps operate to pump fluid, such as blood, by positive displacement using revolving rollers that occlude flexible tubing. With respect to cardiopulmonary bypass machines, multiple roller pumps may be employed as part of a perfusion circuit to provide aortic root suction, vent suction, a blood cardioplegia pump, and a systemic blood pump. However, roller pumps may be employed in other environments, such as with a dialyzer or as a pump to feed intravenous (IV) fluids intravenously to a patient.

Roller pumps are simply structured devices that produce a constant flow, and use disposable tubing as a fluid path through which fluid is pumped. Roller pumps generally include a pump drive and a pump head, wherein the pump drive is connected to drive rotation of the pump head to pump fluid. The pump head includes a pump stator and a pump rotor, wherein the pump stator forms a housing that defines an inner circumferential surface, or raceway, against which one or more tubes through which fluid flows are compressed by rollers connected to the pump rotor. Upon rotation of the pump rotor by rotation of a drive shaft connected to the pump rotor, the rollers connected to the pump rotor compress the fluid tubing against the inner circumference of the pump stator so that rolling of the rollers along the tubing pushes fluid in the tubing in the direction of the pump rotor's rotation. The amount of compression (i.e., occlusion) applied by the pump rollers against the fluid tubing needs to be adjusted to compensate for subtle manufacturing differences of fluid tubing with respect to diameter, for example, provided by different vendors, or to compensate for the use of different sized fluid tubing.

Because the fluid flowing in the tubing may include blood, it is important that the roller pumps be adjustable with respect to the degree of compression of the tubing during roller rotation and pumping. If compression is complete so that the walls of the tubing completely collapse during compression, then red blood cells of the pumped blood may be lysed, thereby rendering the pumped blood less suitable for use. If the compression is inadequate so that the walls of the tubing are not adequately compressed during pump rotor rotation, then inadequate fluid flow or even backflow may result. Therefore, it is necessary to be able to adjust the amount of compression the rollers and the raceway exert on the tubing during pump rotor rotation in view of the fact that fluid tubing of different diameters may be used in the roller pumps.

The ability to adjust the amount of compression exerted by the rollers against the fluid tubing in the raceway is known in the art as “occlusion,” and this term reflects the degree to which the fluid tubing is compressed, or occluded, between the rollers and the raceway surface during rotation of the pump rotor. Roller pumps are generally provided with a mechanism to adjust occlusion by rotating a knob, or turning some other assembly, in one direction in order to move the rollers radially outward and closer to the inner surface of the pump stator, thereby increasing occlusion, and to rotate the knob, or other assembly, in another direction in order to move the rollers radially inward and away from the inner surface of the pump stator, thereby decreasing occlusion. Thus, when the pump rollers are completely disengaged from the fluid tubing in the raceway, there is a need to be able to rapidly move the pump rollers to engage the fluid tubing in the raceway. When the pump rollers have engaged the fluid tubing in the raceway, there is a need to move the rollers in fine increments in order to finely tune the degree of occlusion. Once the degree of occlusion has been fine-tuned, then the increments are locked into portion for the duration of a pumping procedure. Pump roller increments may be evaluated as the gap distance between the roller and corresponding parts of the fluid tubing path. In addition, all of the rollers of the roller pump are required to maintain the same amount of compression of the fluid tubing.

A force is required to compress the fluid tubing to achieve the desired amount of occlusion. The force required by the user to obtain the desired amount of fluid tubing compression is typically directly related to the tube compression force. Prior art actuating mechanisms, such as a thumb wheel actuating mechanisms for instance, for an occlusion adjustment mechanism for a roller pump have poor mechanical advantage and lead to hindered usability including damage to gloves and a high number of wheel adjustments needed. This is undesirable.

The level of occlusion of peristaltic blood pumps must be precisely adjusted to efficiently move the blood inside the tubing without damaging the blood. A high degree of synchronicity of the occlusion rollers is necessary to reduce pressure differences in the outlet pressure. Thereby, a variation of tube diameters are currently used to achieve this result.

A commonly used method to set occlusion in a blood pump involves counting the drop rate of a column of fluid through a tubing loop. The occlusion mechanism of the roller pump is then manually adjusted to achieve a defined drop rate, which correlates to the desired level of occlusion. This method is time consuming and can only be practiced when the pump is stopped. An adjustment during use, e.g. reducing tube consistency by temperature increase, is not attainable.

Most perfusion pumps have a central knob to set the occlusion manually, but in all cases the rollers are moved together. It is not possible to adjust one roller separately to compensate differences in synchronicity between the rollers that may arise due to manufacturing tolerances. This difference can only defined by the precision of the parts itself.

FIG. 1 illustrates a prior art occlusion adjustment mechanism 2 for a roller pump that is integrated into a pump head 3 of the roller pump, and that moves pump rollers in a linear manner. The pump head 3 includes a pump rotor 4 disposed to rotate within the pump stator 6. The pump rotor 4 includes a plurality of pump rollers 8 mounted to a corresponding number of roller blocks 10. The occlusion adjustment mechanism 2 includes a knob 12 that may be rotated manually so as to turn an elongated rod 14. A tapered drive piston 16 is threadingly connected to the elongated rod 14 so that rotation of the elongated rod 14 moves the tapered drive piston 16 along the elongated rod 14. Movement of the tapered drive piston 16 on the elongated rod 14 causes the tapered drive piston 16 to push on the plurality of roller blocks 10 so that the tapered drive piston 16 moves the roller blocks 10 in a radial direction with respect to the central axis of the pump head 3 in a linearly and uniform manner. Thus, the knob 12 of this system is connected to screw threads (not specifically illustrated) of the elongated rod 14 in order to advance a wedge (i.e., the tapered drive piston) that drives the rollers 8 towards the housing circumference, namely, the inner circumferential surface of the stator 6, which defines a tubing raceway. For images showing how tubing may be disposed in the raceway of a roller pump, one may consult U.S. Patent Application Publication No. US 2014/0127063 A1, which is incorporated herein by reference in its entirety. A disadvantage associated with the tapered drive piston 16 is that such a wedge is dependent upon precise machining of the wedge sides, and those components that interface with the wedge sides, in order to maintain position symmetry of the rollers 8.

Therefore, there is a need for a mechanism for adjusting occlusion of a roller pump, such as a roller pump employed as a cardiac pump of a heart-lung machine and like devices, that permits individual actuation of each roller, which provides for adjusting the occlusion created by each roller, and which provides fine adjustment movements of each of the pump rollers independent of one another. This disclosure is directed to describing embodiments of apparatuses and methods pertaining to adjustment of occlusion of a roller pump, and like devices, that employ such an occlusion adjustment mechanism.

### SUMMARY OF THE DISCLOSURE

In one non-limiting embodiment or aspect of the present disclosure, a roller pump may include a pump head operably connected to be driven by a pump drive assembly, wherein the pump head includes: a pump stator comprising an inner circumferential surface, defining a raceway; a pump rotor disposed to rotate within the pump stator, wherein the pump rotor includes one or more rollers, wherein each roller is connected to a respective roller block that is moveable radially with respect to a central axis of the pump head; an occlusion adjustment mechanism, connected to the roller block of each roller, wherein the occlusion adjustment mechanism operates to move each roller block radially with respect to the central axis of the pump head in order to compress and partially occlude a compressible fluid conduit disposed within the raceway; and a sensor arrangement configured to measure a pressure or force exerted by the rollers when the compressible fluid conduit is compressed by one or more of the rollers.

In one non-limiting embodiment or aspect of the present disclosure, an encoder may be configured to compare a pressure or force exerted by the roller block of each roller and measured by the sensor arrangement to determine whether a pressure differential is equal to zero. Upon exceeding the predetermined pressure limit, the encoder may be configured to readjust a position of the roller blocks of the rollers. The sensor arrangement may include at least one pressure sensor configured to measure the pressure or force exerted by the roller block of each roller. An encoder may be configured to receive a pressure or force measurement from each force sensor, wherein the encoder is configured to compare a difference between the pressure or force measurements to a zero differential. A cable passage may be defined in the pump head to receive wiring from the sensor arrangement. A slip ring may be operatively connected between the pump rotor and the pump stator to permit signal transmission and power transmission between the pump rotor and the pump stator. The rollers may be actuated using linear actuators. The linear actuators may be configured to be adjusted based on a pressure or force measured by the sensor arrangement. Each linear actuator may be provided with a wedge member to hold a pressure sensor of the sensor arrangement, and wherein, as the linear actuator is activated, the wedge member is configured to press the pressure sensor against the respective roller block. A screw member may be provided in each linear actuator, and wherein, as the screw member is rotated, the respective wedge member is moved up or down in order to move the pressure sensor towards or away from the respective roller block. The linear actuators may be positioned on a top surface of the pump head. The sensor arrangement may be configured to continuously measure pressure or force exerted by the roller block of each roller. The roller blocks may be configured to press against a fluid tube directed through the pump head to move a fluid through the fluid tube. The sensor arrangement may be operatively connected to a slip ring positioned on the pump head. The sensor arrangement may be operatively connected to a pair of linear actuators that are configured to direct the sensor arrangement towards and away from the roller blocks, and wherein the linear actuators are each operatively connected to an encoder. At least one spring may be provided in the pump head, wherein the at least one spring is configured to direct the roller blocks back to a return position after the sensor arrangement has taken the pressure or force measurement. Each roller block may be independently moveable radially with respect to the central axis of the pump head so that movement and position of each roller is independently adjusted by the occlusion adjustment mechanism based upon pressure or force measurements provided by the sensor arrangement.

In one non-limiting embodiment or aspect of the present disclosure, a method of adjusting occlusion of a compressible fluid conduit within a pump head may include the steps of: disposing a compressible fluid conduit in a raceway of a roller pump head, wherein the raceway is defined by an inner circumferential surface of a stator of the roller pump head, and the fluid conduit includes a lumen through which fluid flows; operating an occlusion adjustment mechanism of the roller pump head so as to move a plurality of roller blocks of the roller pump head in a radial direction with respect to a central axis of the pump head so as to partially occlude the lumen of the compressible fluid conduit by compressing the compressible fluid conduit between a plurality of rollers connected to the plurality of roller blocks; measuring an exerted pressure or force value for each one of the roller blocks of the roller pump using a sensor arrangement positioned within the pump head; and adjusting position of one or more of the rollers based upon the one or more exerted pressure or force values obtained by the sensor arrangement.

In one non-limiting embodiment or aspect of the present disclosure, the method may include comparing a difference between the pressure or force exerted by each of the roller blocks and determining whether the difference is equal to zero. The method may include adjusting the occlusion adjustment mechanism upon the difference not being equal to zero. The occlusion adjustment mechanism may be operable to independently move each roller block of the plurality of plurality of roller blocks so as to independently adjust position of each roller of the plurality of rollers, and the method further includes the step of: independently adjusting the position of each roller based on the measured pressure or force values obtained using the sensor arrangement. The lumen of the compressible fluid conduit may be adapted to receive the fluid comprising blood.

Non-limiting illustrative examples of embodiments of the present disclosure will now be described in the following numbered clauses:
Clause 1: A roller pump, comprising: a pump head operably connected to be driven by a pump drive, wherein the pump head includes: a pump stator comprising an inner circumferential surface, defining a raceway; a pump rotor disposed to rotate within the pump stator, wherein the pump rotor includes one or more rollers, wherein each roller is connected to a respective roller block that is moveable radially with respect to a central axis of the pump head; an occlusion adjustment mechanism, connected to the roller block of each roller, wherein the occlusion adjustment mechanism operates to move each roller block radially with respect to the central axis of the pump head in order to compress and partially occlude a compressible fluid conduit disposed within the raceway; and a sensor arrangement configured to measure a pressure or force exerted by the roller block of at least one roller when the compressible fluid conduit is compressed by one or more of the rollers.
Clause 2: The roller pump of Clause 1, further comprising an encoder configured to compare a pressure or force exerted by the roller block of each roller and measured by the sensor arrangement to determine whether a pressure differential is equal to zero.
Clause 3: The roller pump of Clause 2, wherein, upon exceeding the predetermined pressure or force limit, the encoder is configured to readjust a position of the roller blocks of the rollers.
Clause 4: The roller pump of any of Clauses 1-3, wherein the sensor arrangement comprises at least one pressure sensor configured to measure the pressure or force exerted by the roller block of each roller.
Clause 5: The roller pump of Clause 4, further comprising an encoder configured to receive a pressure or force measurement from each force sensor, wherein the encoder is configured to compare a difference between the pressure or force measurements to a zero differential.
Clause 6: The roller pump of any of Clauses 1-5, wherein a cable passage is defined in the pump head to receive wiring from the sensor arrangement.
Clause 7: The roller pump of any of Clauses 1-6 further comprising a slip ring operatively connected between the pump rotor and the pump stator to permit signal transmission and power transmission between the pump rotor and the pump stator.
Clause 8: The roller pump of any of Clauses 1-7, wherein the rollers are actuated using linear actuators.
Clause 9: The roller pump of Clause 8, wherein the linear actuators are configured to be adjusted based on a pressure or force measured by the sensor arrangement.
Clause 10: The roller pump of Clause 8, wherein each linear actuator is provided with a wedge member to hold a pressure sensor of the sensor arrangement, and wherein, as the linear actuator is activated, the wedge member is configured to press the pressure sensor against the respective roller block.
Clause 11: The roller pump of Clause 10, wherein a screw member is provided in each linear actuator, and wherein, as the screw member is rotated, the respective wedge member is moved up or down in order to move the pressure sensor towards or away from the respective roller block.
Clause 12: The roller pump of Clause 8, wherein the linear actuators are positioned on a top surface of the pump head.
Clause 13: The roller pump of any of Clauses 1-12, wherein the sensor arrangement is configured to continuously measure pressure or force exerted by the roller block of each roller.
Clause 14: The roller pump of any of Clauses 1-13, wherein the roller blocks are configured to press against a fluid tube directed through the pump head to move a fluid through the fluid tube.
Clause 15: The roller pump of any of Clauses 1-14, wherein the sensor arrangement is operatively connected to a slip ring positioned on the pump head.
Clause 16: The roller pump of any of Clauses 1-15, wherein the sensor arrangement is operatively connected to a pair of linear actuators that are configured to direct the sensor arrangement towards and away from the roller blocks, and wherein the linear actuators are each operatively connected to an encoder.
Clause 17: The roller pump of any of Clauses 1-16, further comprising at least one spring provided in the pump head, wherein the at least one spring is configured to direct the roller blocks back to a return position after the sensor arrangement has taken the pressure or force measurement.
Clause 18: The roller pump of any of Clauses 1-17, wherein each roller block is independently moveable radially with respect to the central axis of the pump head so that movement and position of each roller is independently adjusted by the occlusion adjustment mechanism based upon pressure or force measurements provided by the sensor arrangement.
Clause 19: A method of adjusting occlusion of a compressible fluid conduit within a pump head, the method comprising the steps of: disposing a compressible fluid conduit in a raceway of a roller pump head, wherein the raceway is defined by an inner circumferential surface of a stator of the roller pump head, and the fluid conduit includes a lumen through which fluid flows; operating an occlusion adjustment mechanism of the roller pump head so as to move a plurality of roller blocks of the roller pump head in a radial direction with respect to a central axis of the pump head so as to partially occlude the lumen of the compressible fluid conduit by compressing the compressible fluid conduit between a plurality of rollers connected to the plurality of roller blocks; measuring an exerted pressure or force value for each one of the roller blocks of the roller pump using a sensor arrangement positioned within the pump head; and adjusting position of one or more of the rollers based upon the one or more exerted pressure or force values obtained by the sensor arrangement.
Clause 20: The method of Clause 19, comparing a difference between the pressure or force exerted by each of the roller blocks and determining whether the difference is equal to zero.
Clause 21: The method of Clause 19 or Clause 20, adjusting the occlusion adjustment mechanism upon the difference not being equal to a zero differential.
Clause 22: The method of any of Clauses 19-21, wherein the occlusion adjustment mechanism is operable to independently move each roller block of the plurality of plurality of roller blocks so as to independently adjust position of each roller of the plurality of rollers, and the method further includes the step of: independently adjusting the position of each roller based on the measured pressure or force values obtained using the sensor arrangement.
Clause 23: The method of any of Clauses 19-22, wherein the lumen of the compressible fluid conduit is adapted to receive the fluid comprising blood.

These and other features and characteristics of the novel technology, as well as the methods of operation and functions of the related elements of structures and the combination of parts and economies of manufacture, will become more apparent upon consideration of the following description and appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure or any invention. As used in the specification and the claims, the singular form of “a”, “an”, and “the” include plural referents unless the context clearly dictates otherwise.

### BRIEF DESCRIPTION OF THE DRAWINGS

FIG. 1 is a cross-sectional view of a prior art occlusion adjustment mechanism that adjusts roller occlusion in a linear manner.

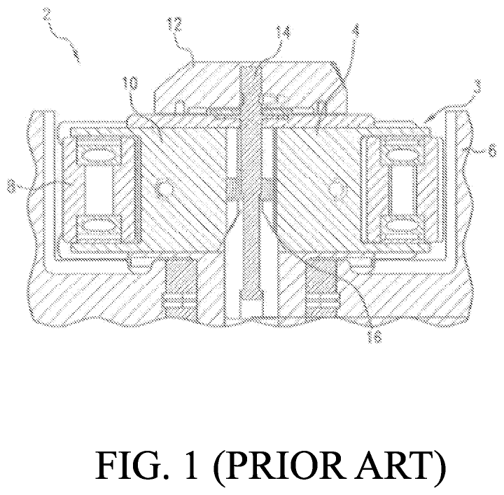

FIG. 2 is a perspective view of a pump head according to one non-limiting embodiment or aspect of the present disclosure.

FIG. 3 is a cross-sectional view of the pump head of FIG. 2 illustrating an occlusion adjustment mechanism that adjusts roller occlusion in a linear manner in which the rollers are in a pre-applied position.

FIG. 4 is a cross-sectional view of the pump head of FIG. 2 illustrating an occlusion adjustment mechanism that adjusts roller occlusion in a linear manner in which the rollers are in an intermediate position.

FIG. 5 is a cross-sectional view of the pump head of FIG. 2 illustrating an occlusion adjustment mechanism that adjusts roller occlusion in a linear manner in which the rollers are in an applied position.

FIG. 6 is a cross-sectional view of the pump head of FIG. 2.

### DESCRIPTION OF NON-LIMITING EMBODIMENTS OF THE DISCLOSURE

The illustrations generally show illustrative and non-limiting aspects of the devices, assemblies, and methods of the present disclosure. While the descriptions present various aspects of the devices and assemblies, it should not be interpreted in any way as limiting the disclosure. Furthermore, modifications, concepts, and applications of the disclosure's aspects are to be interpreted by those skilled in the art as being encompassed by, but not limited to, the illustrations and descriptions herein.

Further, for purposes of the description hereinafter, the terms “end”, “upper”, “lower”, “right”, “left”, “vertical”, “horizontal”, “top”, “bottom”, “lateral”, “longitudinal”, “radial”, and derivatives thereof shall relate to the disclosure as it is oriented in the drawing figures. The term “proximal” refers to an end of the device that is configured to be manipulated by a user or, for an implanted device, the side or end of the device that remains closest to the implantation site, when the device is deployed. The term “distal” refers to the end of the device opposite from the proximal end, which can be the end of the device farthest away from portions of the device intended to be manipulated by a user. For an implanted device, the “distal” end of the device is the end of the device farthest away from the implantation site. However, it is to be understood that the disclosure may assume various alternative variations and step sequences, except where expressly specified to the contrary. It is also to be understood that the specific devices and processes illustrated in the attached drawings, and described in the following specification, are simply exemplary aspects of the disclosure. Hence, specific dimensions and other physical characteristics related to the aspects disclosed herein are not to be considered as limiting. For the purpose of facilitating understanding of the disclosure, the accompanying drawings and description illustrate preferred aspects thereof, from which the disclosure, various aspects of its structures, construction and method of operation, and many advantages may be understood and appreciated.

For the purposes of this disclosure, the term “about” refers to a range of ±10% of the stated value.

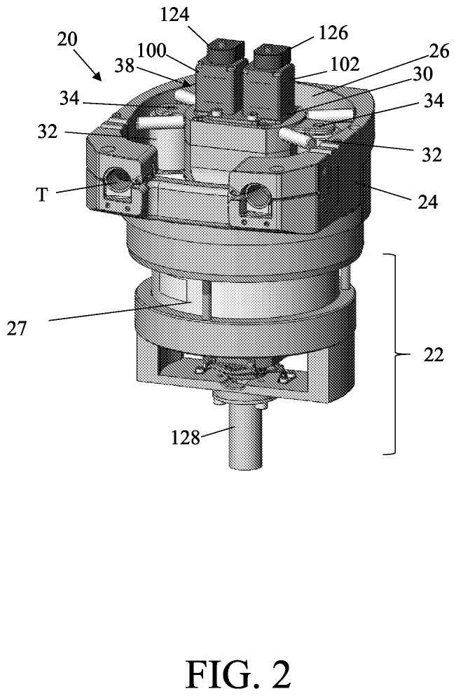

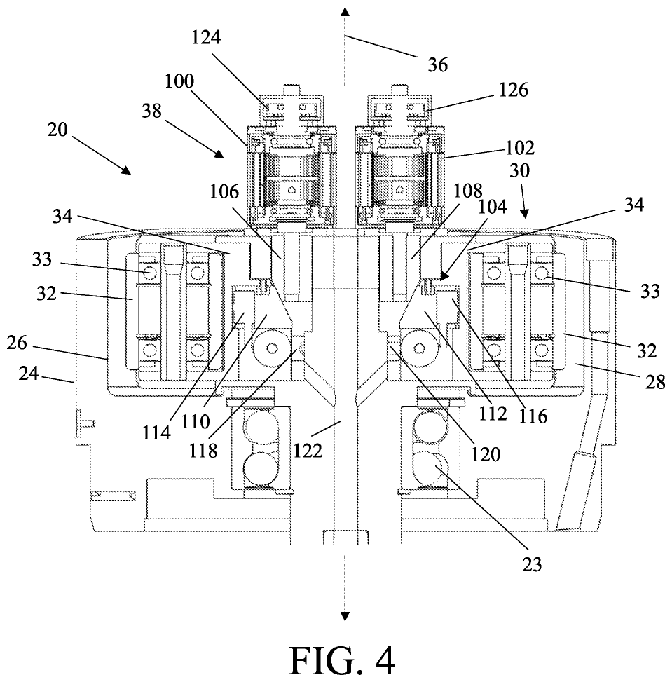

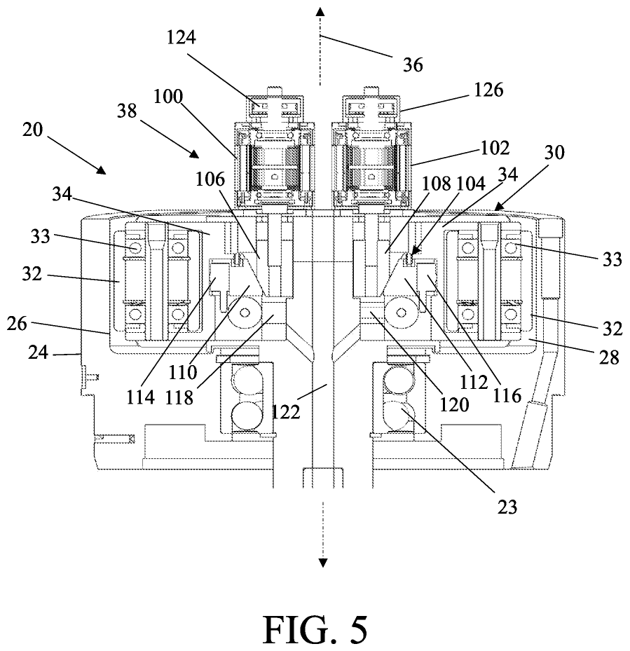

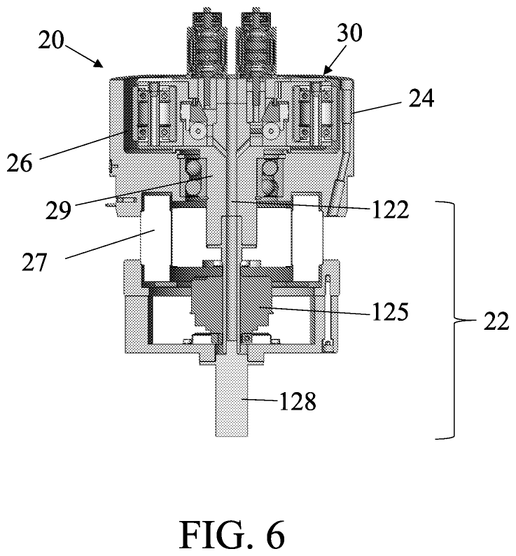

As shown in FIGS. 2 and 3-6, a pump head 20 of a roller pump is operably connected to be driven by a pump drive assembly 22 of the roller pump, the pump drive assembly 22 including a pump motor 27 to effect rotation of a pump rotor 30. The pump head 20 includes a pump stator 24 that is provided with an inner circumferential surface 26 defining a raceway 28 for fluid tubing T (See FIG. 2), and a pump rotor 30 disposed to rotate within the pump stator 24. The pump rotor 30 includes a plurality of rollers 32, most typically two rollers for roller pumps used in cardiopulmonary bypass machines although pump heads having three, six or eight rollers, etc., may also be used. Each roller 32 includes roller bearings 33 to allow it to rotate. The pump rotor 30 includes a plurality of roller blocks 34 so that each roller 32 is connected to a respective roller block 34 that is moveable radially with respect to a central axis 36 of the pump head 20. The pump head 20 also includes an occlusion adjustment mechanism 38 connected to the drive shaft 29 (shown in FIG. 6). The occlusion adjustment mechanism 38 may be construed as an integral component of the pump head 20, and operates to move each roller block 34 radially with respect to the central axis 36 of the pump head in a non-linearly non-uniform manner. The pump head 20 may also include a bearing 23 that permits the rollers 32 and occlusion adjustment mechanism 38 components to rotate relative to the stator 24.

The occlusion adjustment mechanism 38 is oriented on the central axis 36 of the pump head 20. The range of movement of the rollers 32 extends between a position in which the rollers 32 are fully retracted in which the gap of the raceway 28 is maximal, and a position in which the rollers 32 are positioned in a fully extended position towards the inner circumferential surface 26 of the stator 24 in which the gap of the raceway 38 is minimal.

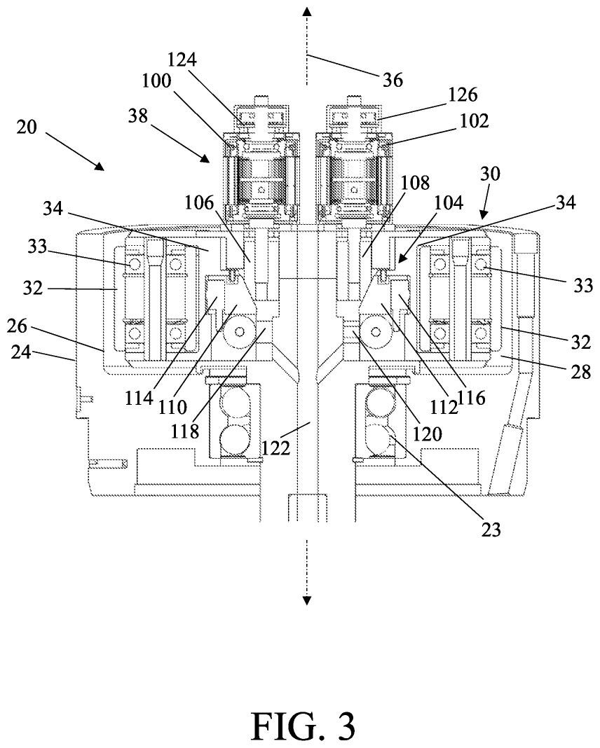

With reference to FIGS. 3-6, in accordance with one embodiment or aspect, the pump head 20 may include at least two actuators 100, 102 for adjusting the position of the rollers 32 provided in the pump 20. A sensor arrangement 104 is operatively connected to each roller arrangement and may be a force feedback sensor arrangement to measure a force exerted by the rollers 32 on the tubing T. It is to be understood that, while two actuators 100, 102 are illustrated in use with the pump head 20, it is also contemplated that fewer or additional actuators may be provided to assist in the force feedback measurements of the sensor arrangement 104. In one embodiment, the actuators 100, 102 are operatively connected to a top surface of the pump head 20. In one embodiment, the actuators 100, 102 may be linear actuators including a lead screw that can extend from and retract within the actuator 100, 102.

In one embodiment or aspect, each actuator 100, 102 may be operatively connected to a wedge member 106, 108. The lead screw of each actuator 100, 102 may be operatively connected to the respective wedge member 106, 108. As the lead screw of each actuator 100, 102 is rotated in a first direction (for example, in a clockwise direction), the respective wedge member 106, 108 may be moved in a downward direction away from the respective actuator 100, 102. As the lead screw of each actuator 100, 102 is rotated in a second direction (for example, in a counter-clockwise direction), the respective wedge member 106, 108 may be moved in an upward direction towards the respective actuator 100, 102.

In one embodiment or aspect, the movement of the wedge members 106, 108 is configured to effect movement of a respective sensor holder 110, 112 held in the pump head 20 adjacent or in close proximity to the tubing T. The pump head 20 may include at least two sensor holders 110, 112 adjacent to or in close proximity to the tubing T and on opposing sides of the pump head 20. Each sensor holder 110, 112 may be sized and configured to retain a corresponding force sensor 114, 116 in a cavity defined in each sensor holder 110, 112. An inner surface (a surface closest to the center of the pump head 20) of each sensor holder 110, 112 may be inclined or angled relative to a central axis 36 of the pump head 20. In one embodiment or aspect, a diameter of a top portion of each sensor holder 110, 112, may be smaller than a diameter of a bottom portion of each sensor holder 110, 112, such that the diameter of the sensor holder 110, 112, expands from the top of the sensor holder 110, 112 to the bottom of the sensor holder 110, 112. As the lead screw of each actuator 100, 102 moves each respective wedge member 106, 108, the wedge member 106, 108 presses against the inclined or angled inner surface of the respective sensor holder 110, 112. As the wedge member 106, 108 slides along the inclined or angled inner surface of the respective sensor holder 110, 112, the sensor holder 110, 112 is pushed or moved towards the rollers 32 to bring the force sensors 114, 116 in operative contact with the rollers 32 and to move the rollers 32 in an outwardly radial direction. Once the force sensors 114, 116 have been moved adjacent or in contact with the rollers 32, the force sensors 114, 116 may be configured to measure the force with which the occlusion rollers 32 press against the tubing T.

In one embodiment, an encoders 124, 126 operatively connected (either wired or wirelessly) to the pump head 20, and in one example, operatively connected to the actuators 100, 102, may compare the measurements being recorded by the force sensors 114, 116. The force values of both force sensors 114, 116 may be compared and, in the event there is a difference higher than a defined limit, the actuators 100, 102 will readjust the position of the occlusion rollers 32. Using this sensor arrangement, the occlusion (a distance between the rollers 32 and raceway 28) can be precisely adjusted. After the force sensors 114, 116 have provided the desired force measurements, the lead screw of each actuator 100, 102 may be rotated in an opposing direction to move the wedge members 106, 108 (and the force sensors 114, 116) away from the occlusion rollers 32. As the lead screw is continually rotated, a spring 118, 120 operatively connected to one of the respective wedge members 106, 108 are configured to assist in moving the wedge member 106, 108 away from the sensor holders 110, 112.

With reference to FIGS. 3-5, according to one embodiment or aspect of the present disclosure, the pump head 20 may also include a passageway 122 that extends between a top portion and a bottom portion of the pump head 20. The passageway 122 may be configured to receive and hold cables that connect the force sensors 114, 116, the linear actuators 100, 102, and encoders 124, 126 each operatively connected to one of the linear actuators 100, 102, and an encoder 125 provided in the pump drive assembly 22. The cables of the force sensors 114, 116, the linear actuators, 100, 102, and the encoders 124, 126 may be connected to a slip ring 128 positioned and held in the passageway 122. The encoders 124, 126 are provided to send information to a controller (not illustrated), the information being recorded by the encoders 124, 126 being directed to the rotation angle of the pump rotor 4, as well as how fast the pump rotor 4 is rotating.

While apparatuses and methods have been described with reference to certain embodiments within this disclosure, one of ordinary skill in the art will recognize, that additions, deletions, substitutions and improvements can be made while remaining within the scope and spirit of the invention is defined by the appended claims.

1. A roller pump, comprising:
a pump head operably connected to be driven by a pump drive assembly, wherein the pump head includes: a pump stator comprising an inner circumferential surface, defining a raceway; a pump rotor disposed to rotate within the pump stator, wherein the pump rotor includes one or more rollers, wherein each roller is connected to a respective roller block, and wherein each roller block is independently moveable radially with respect to a central axis of the pump head;
an occlusion adjustment mechanism, operatively connected to the roller block of each roller, wherein the occlusion adjustment mechanism operates to move each roller block radially with respect to the central axis of the pump head in order to compress and partially occlude a compressible fluid conduit disposed within the raceway; and
a sensor arrangement configured to measure a pressure or force exerted by the rollers when the compressible fluid conduit is compressed by one or more of the rollers.

2. The roller pump of claim 1, further comprising an encoder configured to compare a pressure or force exerted by the each of the rollers and measured by the sensor arrangement to determine whether the pressure or force is greater than a zero differential.

3. The roller pump of claim 2, wherein, upon exceeding the zero differential, the encoder is configured to direct the occlusion adjustment mechanism to readjust a position of the roller blocks of the rollers.

4. The roller pump of claim 1, wherein the sensor arrangement comprises at least one pressure sensor configured to measure the pressure or force exerted by each roller individually.

5. The roller pump of claim 4, further comprising an encoder configured to receive a pressure or force measurement from each force sensor, wherein the encoder is configured to compare a difference between the pressure or force measurements to a zero differential.

6. The roller pump of claim 1, wherein a cable passage is defined in the pump head to receive wiring from the sensor arrangement.

7. The roller pump of claim 1, further comprising a slip ring operatively connected between the pump rotor and the pump stator to permit signal transmission and power transmission between the pump rotor and the pump stator.

8. The roller pump of claim 1, wherein the rollers are actuated using linear actuators.

9. The roller pump of claim 8, wherein the linear actuators are configured to be adjusted based on a pressure or force measured by the sensor arrangement.

10. The roller pump of claim 8, wherein each linear actuator is provided with a wedge member to hold a pressure sensor of the sensor arrangement, and wherein, as the linear actuator is activated, the wedge member is configured to press the pressure sensor against the respective roller block.

11. The roller pump of claim 10, wherein a screw member is provided in each linear actuator, and wherein, as the screw member is rotated, the respective wedge member is moved up or down in order to move the pressure sensor towards or away from the respective roller block.

12. The roller pump of claim 8, wherein the linear actuators are positioned on a top surface of the pump head.

13. The roller pump of claim 1, wherein the sensor arrangement is configured to continuously measure pressure or force exerted by each roller.

14. The roller pump of claim 1, wherein the rollers are configured to press against a fluid tube directed through the pump head to move a fluid through the fluid tube.

15. The roller pump of claim 1, wherein the sensor arrangement is operatively connected to a slip ring positioned on the pump head.

16. The roller pump of claim 1, wherein the sensor arrangement is operatively connected to a pair of linear actuators that are configured to direct the rollers towards and away from the inner circumferential surface, and wherein the linear actuators are each operatively connected to an encoder.

17. The roller pump of claim 1, further comprising at least one spring provided in the pump head, wherein the at least one spring is configured to direct the roller blocks back to a return position after the sensor arrangement has taken the pressure or force measurement.

18. The roller pump of claim 1, wherein each roller block is independently moveable radially with respect to the central axis of the pump head so that movement and position of each roller is independently adjusted by the occlusion adjustment mechanism based upon pressure or force measurements provided by the sensor arrangement.

19. A method of adjusting occlusion of a compressible fluid conduit within a pump head, the method comprising the steps of:
disposing a compressible fluid conduit in a raceway of a roller pump head, wherein the raceway is defined by an inner circumferential surface of a stator of the pump head, and the fluid conduit includes a lumen through which fluid flows;
operating an occlusion adjustment mechanism of the roller pump head so as to move a plurality of roller blocks of the pump head independently in a radial direction with respect to a central axis of the pump head so as to partially occlude the lumen of the compressible fluid conduit by compressing the compressible fluid conduit between a plurality of rollers connected to the plurality of roller blocks;
measuring an exerted pressure or force value for each one of the rollers of the roller pump using a sensor arrangement positioned within the pump head; and
adjusting position of one or more of the rollers based upon the one or more exerted pressure or force values obtained by the sensor arrangement.

20. The method of claim 19, comparing a difference between the pressure or force exerted by each of the rollers and determining whether the difference exceeds a zero differential.
